(19) United States
(12) Patent Application Publication
(10) Pub. No.: US 2026/0192069 A1
(43) Pub. Date: Jul. 9, 2026

(54) OXYGENATING OROPHARYNGEAL AIRWAY DEVICES, KITS RELATING TO SAME, AND METHODS OF USING SAME

(71) Applicant: BAYLOR RESEARCH INSTITUTE D/B/A BAYLOR SCOTT & WHITE RESEARCH INSTITUTE, Dallas, TX (US)

(72) Inventor: Jeramie HANSON, Belton, TX (US)

(21) Appl. No.: 19/130,679

(22) PCT Filed: Nov. 16, 2023
(86) PCT No.: PCT/US2023/080124

§ 371 Date: May 16, 2025

Related U.S. Application Data
(60) Provisional application No. 63/426,055, filed on Nov. 17, 2022.

(51) Int. Cl. A61M 16/04; A61M 16/08

(52) U.S. Cl. CPC A61M 16/0431; A61M 16/0463; A61M 16/0486; A61M 16/0816; A61M 2202/0208

(57) ABSTRACT

Oxygenating oropharyngeal airway devices, kits including at least such devices, and methods of using such devices. Such a device may be coupled to a host oropharyngeal airway, such as a Berman-style airway, inserted into a patient in need of airway management, uncoupled from the host airway, and used to oxygenate the patient through a lumen of the device both before and during any needed intubation. Another lumen of the device may be used to monitor end tidal carbon dioxide. A spray catheter may also be used in conjunction with the device to topicalize a patient's airway blindly. Ventilation, achieved by manipulating a bag coupled to a mask adequately sealed over the patient's nose and mouth, may be performed while one of the devices is positioned in the patient's mouth and while the patient is receiving oxygen from tubing running under the mask and attached to the device and an oxygen source.

This application claims the benefit of priority of U.S. Provisional Patent Application No. 63/426,055, filed Nov. 17, 2022, which is hereby incorporated by reference in its entirety.

### FIELD OF INVENTION

The invention relates generally to oral airway devices that can be inserted into the airway of a living being for the purpose of facilitating oxygenation. The invention also relates to kits that include one or more such devices, and to methods of using such devices.

### BACKGROUND

High-flow nasal cannula (HFNC) oxygen therapy was initially introduced in the early 2000s for management of apnea in neonates and has recently gained favor in the adult operating room and critical care settings. Through anatomic dead space washout and improved positive end expiratory pressure, HFNC oxygen therapy can effectively allow maintenance of normal oxygen saturations in apneic patients for prolonged periods of time. HFNC oxygen therapy does, however, have several limiting factors. First, it requires additional equipment, including an oxygen heater, a humidifier, a high-flow oxygen regulator, and a nasal cannula designed for this purpose. Additionally, HFNC oxygen therapy is relatively inefficient, requiring oxygen flow rates up to sixty (60) liters per minute. Finally, because of the additional equipment required, HFNC oxygen therapy is not ideal in emergent situations.

### SUMMARY OF THE INVENTION

Embodiments of the present oxygenating oropharyngeal airway devices, which may be referred to as oral airway devices, can be used to provide relatively high-flow oxygen orally, directly above the glottis. Supraglottic oxygenation with embodiments of the present oral airway devices may be achieved during ventilation and during intubation. It is believed that, by bypassing the dead space and soft tissue obstruction of the nasopharynx using one of these embodiments, maintenance of normal oxygen saturation can be achieved at oxygen flow rates lower than sixty liters per minute (in at least some circumstances) and without the need for an oxygen heater, a humidifier, or a high-flow oxygen regulator. Embodiments of the present oral airway devices include a connector configured to be coupled to an oxygen source (such as a wall oxygen source or an e-cylinder), making them suitable for the operating room, emergency department, and critical-care settings. That connector is in fluid communication with a lumen within the oral airway device through which oxygen from an oxygen source may be directed to provide apneic oxygenation to the patient.

The present oral airway devices are configured to fit within a host airway device, such as a channel of a “Berman” oropharyngeal airway. As a result, both such airway devices—in their coupled state—can be placed in the patient's mouth at the same time. Once the practitioner is ready to intubate the patient, the practitioner may uncouple the two devices while they are still in the patient's mouth and remove the host airway device from the patient's mouth. The present oral airway device may remain in the left side of the mouth and be used to continue providing passive oxygenation during laryngoscopy and intubation.

Embodiments of the present oral airway devices include an additional lumen (not in fluid communication with the oxygen lumen) through which an elongated member, such as a catheter with multiple openings along a distal portion thereof, may be inserted and used to spray a local anesthetic (such as lidocaine) into the patient to topicalize the patient's airway. In contrast to airway topicalization requiring the practitioner to perform direct laryngoscopy and use a laryngotracheal-administered device, a practitioner using such embodiments of the present oral airway devices will have the option to topicalize the patient's airway blindly, without the need for airway instrumentation. To facilitate such topicalization, the embodiments of the present oral airway devices and associated catheters may be sized relative to the patient so that the distal end of the catheter may be extended to a location within the patient that is under the epiglottis.

Embodiments of the present oral airway devices also include a connector, in fluid communication with the lumen through which the spray catheter may be inserted, that can be configured as a port connectable to tube that can be used as a carbon dioxide (CO₂) sample line to monitor end tidal CO₂, which may be referred to as ETCO₂ for ease of reference.

Embodiments of the present oral airway devices can be utilized in a variety of different situations and deployed quickly in urgent and emergency situations. The present oral airway devices can serve to oxygenate patients requiring cardio pulmonary resuscitation (CPR), thereby limiting interruptions in chest compressions in the non-intubated patient and enabling the monitoring of ETCO₂, which will reflect whether quality chest compressions are being performed. The present oral airway devices may also serve as an effective emergency airway adjunct in situations where a patient cannot be ventilated.

Embodiments of the present methods include any one or more of the uses of an embodiment of the present oral airway devices described above. Accordingly, in some embodiments of the present methods, one of the present oral airway devices may be coupled to a host oropharyngeal airway device and inserted into a patient's mouth, including a patient undergoing CPR or who undergoes CPR after such insertion. In some embodiments of the present methods, one of the present oral airway devices can be used to blindly topicalize a patient's airway and vocal cords without being impeded by soft tissue. In some embodiments of the present methods, oxygen may be delivered to a patient at a rate of five (5) to twenty-five (25) liters per minute (including every measurable increment of liters per minute between five and twenty-five, including increments of one) through one of the present oral airway devices that has been inserted into the patient's mouth. In some embodiments of the present methods, one of the present oral airway devices may be used to apply positive end-expiratory pressure (PEEP) to a patient to prevent, reverse, or improve atelectasis. The present oral airway devices are configured such that, when the device is connected to a tube (that is connected to an oxygen source) and/or to a tube that is connected to equipment for monitoring end tidal carbon dioxide, a conventional mask that can be used for bag-valve-mask ventilation may be placed over the patient's nose and mouth and adequately sealed; as a result, some embodiments of the present methods include placing a mask over the patient's nose and mouth while one of the present devices (connected, or not, to one or more tubes) is inserted in the patient's mouth and ventilating the patient using a bag coupled to the mask.

The present kits may include any one or more of: one of the present oral airway devices; a host airway device that is sized for use therewith and that may be coupled thereto; a length of oxygen tubing with connectors, one of which is configured for connection to an oxygen source and the other of which is configured for connection to the oxygen connector of the included one of the present oral airway devices (such as to the female oxygen tubing connector on a shorter length of oxygen tubing attached to the oxygen connector of the included one of the present oral airway devices); and one of the present spray catheters that has been sized for use with the included one of the present oral airway devices and (optionally) inserted into a lumen thereof. At least some embodiments of the present kits includes a container, such as a box or a pouch, that is sealed and includes any one or more of the foregoing components. In at least some embodiments of the present kits that include one of the present oral airway devices and a comparably-sized host oral airway device, the two airway devices may have colors that reflect their sizes (e.g., pink for an infant) and that they are suited (in size) for use together but that are not identical to each other so that a practitioner is capable of distinguishing the two devices when they are coupled together; any spray catheter that is included with the kit may have a color that matches or is similar to the color of the included one of the present oral airway devices to reflect that the catheter is suited (in size) for use therewith. The items in such a kit may be made from materials that render them suited for a single-use application.

The term “coupled” is defined as connected, although not necessarily directly, and not necessarily mechanically. The terms “a” and “an” are defined as one or more unless this disclosure explicitly requires otherwise. The term “substantially” is defined as largely but not necessarily wholly what is specified (and includes what is specified; e.g., substantially perpendicular includes perpendicular), as understood by a person of ordinary skill in the art. In any disclosed embodiment, the term “substantially” or “approximately” can be substituted with “within [a percentage] of” what is specified, where the percentage includes 0.1, 1, 5, and up to 10 percent.

The phrase “and/or” means and or or. To illustrate, A, B, and/or C includes: A alone, B alone, C alone, a combination of A and B, a combination of A and C, a combination of B and C, or a combination of A, B, and C. In other words, “and/or” operates as an inclusive or.

The terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, something that “comprises,” “has,” “includes,” or “contains” one or more elements possesses those one or more elements, but is not limited to possessing only those one or more elements. Likewise, a method that “comprises,” “has,” “includes,” or “contains” one or more steps possesses those one or more steps, but is not limited to possessing only those one or more steps.

Any embodiment of any of the disclosed oral airway devices and kits and methods can consist of or consist essentially ofrather than comprise/have/include/contain-any of the described elements, features, and/or steps. Thus, in any of the claims, the term “consisting of′ or “consisting essentially of” can be substituted for any of the open-ended linking verbs recited above, in order to change the scope of a given claim from what it would otherwise be using the open-ended linking verb.

Further, an oral airway device or portion thereof that is configured in a certain way is configured in at least that way, but it can also be configured in a way or ways other than those specifically described.

The feature or features of one embodiment may be applied to other embodiments, even though not described or illustrated, unless expressly prohibited by this disclosure or the nature of the embodiments.

Some details associated with some of the embodiments of the present oral airway devices, the present kits, and the present methods are described above, and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears, and every reference number in a given figure is not always described. Identical reference numbers do not necessarily indicate an identical structure. Rather, the same reference number may be used to indicate a similar feature or a feature with similar functionality, as may non-identical reference numbers. The devices in FIGS. 1A-13 are drawn to scale, meaning the sizes of the depicted elements are accurate relative to each other for at least the depicted embodiments of the present oral airway devices and present spray catheters; but as a person of ordinary skill in the art will understand, this invention is not limited to the depicted embodiments or usage of any of them, and other embodiments that do not possess the depicted sizes also fall within the scope of the claims. The devices shown in the figures were drawn (or prototyped, for those shown in FIGS. 14-16) using Shapr3D software and, as a result, include many lines that, as those of ordinary skill in the art will understand, do not represent “hard” changes of angle. Instead, such lines may respectively show where a rounded section meets a flat, or straight, section. Additionally, some lines are extraneous (or show relatively small features or shapes that are extraneous) to the depicted device and are generally the result of expediently generating a three-dimensional printed prototype. The relevant figures were created using the “orthographic” (rather than the “perspective”) “appearance” setting of the software.

FIGS. 1A and 1B are perspective views of one embodiment of the present oral airway devices; the views are from different perspectives.

FIG. 2A is a front view of the device shown in FIGS. 1A and 1B.

FIG. 2B is a back view of the device shown in FIGS. 1A and 1B.

FIG. 3A and FIG. 3B are right and left side views, respectively, of the device shown in FIGS. 1A and 1B.

FIG. 4 shows an enlarged detail perspective view of the distal end of the body of the device shown in FIGS. 1A and 1B.

FIG. 5 is a cross-sectional view of the device shown in FIGS. 1A and 1B taken across a plane that is parallel to the straight section of the body thereof, and showing an interior of the connector and related lumen for conveying oxygen through the device.

FIG. 6 is a cross-sectional view of the device shown in FIGS. 1A and 1B taken across a plane that is parallel to the straight section of the body thereof, and showing an interior of the connector and related lumen through which a spray catheter may be inserted or ETCO2 may be monitored.

FIG. 7 is a perspective view of the device shown in FIGS. 1A and 1B coupled to a host oropharyngeal airway device.

FIG. 8 is a perspective view depicting the device shown in FIGS. 1A and 1B beside one embodiment of one of the present spray catheters.

FIG. 9 is an enlarged detail view of the distal portion of the spray catheter depicted in FIG. 9, showing multiple openings therealong.

FIG. 10 is a cross-sectional view of a distal portion of the spray catheter depicted in FIG. 8, showing an interior of the spray catheter's lumen and the positions of certain openings communicating with the lumen.

FIG. 11 is a perspective view of the device shown in FIGS. 1A and 1B with the spray catheter of FIG. 8 inserted into and through the smaller of the device's two lumens.

FIG. 12 is an enlarged detail view of proximal portions of the two devices shown in FIG. 11.

FIG. 13 is an enlarged detail view of distal portions of the two devices shown in FIG. 11.

FIG. 14 shows one embodiment of the present oral airway devices with a first length of oxygen tubing connected to the device's oxygen connector, and a second length of oxygen tubing that may be connected to an oxygen source (at one end thereof) and to the first length of oxygen tubing (at the other end thereof).

FIG. 15 shows a mask usable with a bag-valve-mask ventilation system (not shown) being placed over a mannequin that has one of the present oral airway devices (coupled to a host oral airway device) inserted in the mannequin's mouth.

FIG. 16 shows the mask from FIG. 15 in a sealed positioned over the depicted oral airway device and the length of tubing attached to a connector thereof, with the length of tubing positioned so that one portion is under the mask and another portion is outside the mask, with the mask adequately sealing for ventilation thereover.

### DETAILED DESCRIPTION

The present oral airway devices are configured to be coupled to a host oropharyngeal airway device (sometimes referred to as an “oropharyngeal airway” or an “OPA”) and inserted into a patient's mouth with the host device for use in a variety of airway-management circumstances, including those involving CPR. The present oral airway devices are also configured so that, while the two devices are still in the patient's mouth, they can be uncoupled from each other and the host airway device (which may be a Berman-type airway) may be removed from the patient's mouth, allowing the present oral airway device to remain in the patient's mouth, including during any subsequent intubation. During use of at least some embodiments of the present oral airway devices, the patient's airway may be blindly topicalized and/or oxygen may be delivered to the patient without the need for the type of equipment typically used in HFNC oxygen therapy and (it is believed) at lower flow rates than are typically associated with such therapy. Furthermore, using one of the present oral airway devices during CPR can allow a non-intubated patient to by oxygenated as chest compressions are performed and, when the device is connected to an ETCO2 monitor, provide evidence of the quality of those compressions.

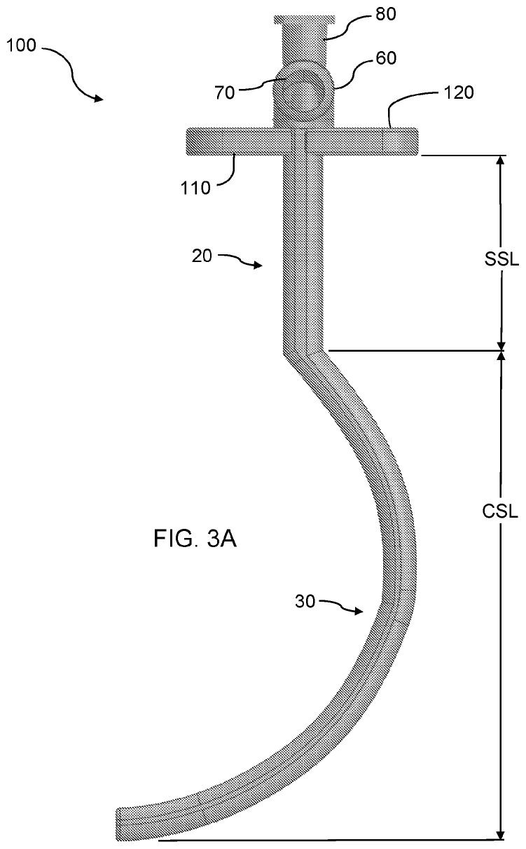

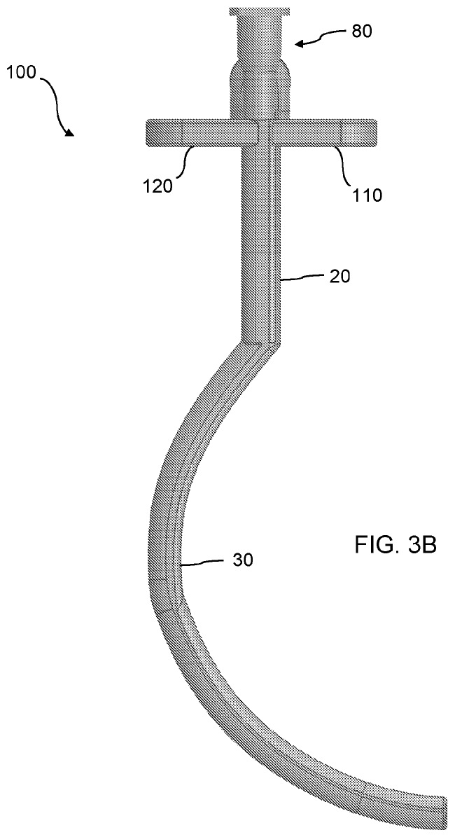

Embodiments of the present oral airway devices may be sized for use in pediatric patients (e.g., those requiring a 40-millimeter (mm) or 43-mm Berman airway) up to extra-large adult patients (e.g., those requiring a 110-mm Berman airway), and thus may be used host airway devices having any suitable lengths, including 43 mm, 60 mm, 80 mm, 90 mm, 100 mm, and 110 mm. These lengths are measured in the direction of the lengths SSL and CSL shown below in FIG. 3A.

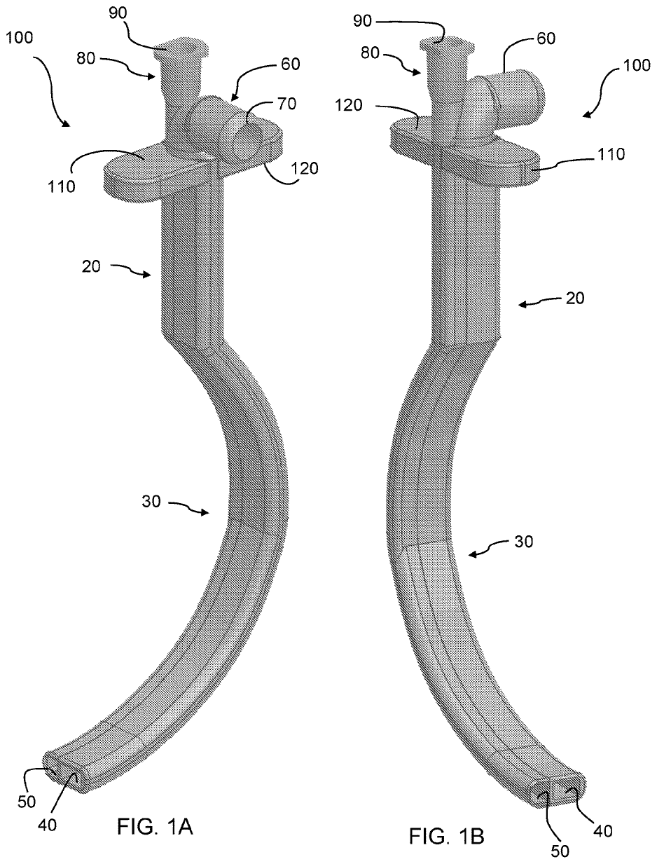

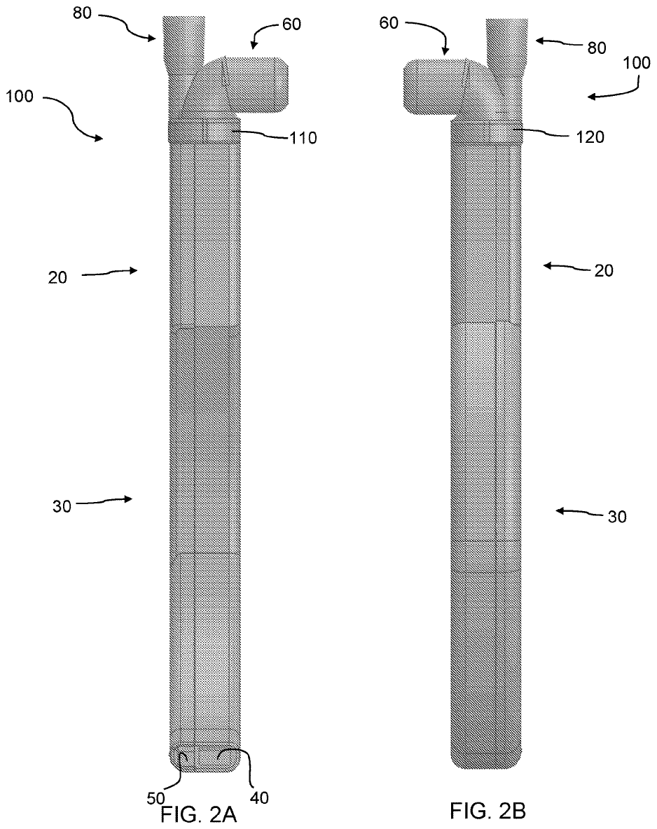

FIGS. 1A and 1B are perspective views of one embodiment of the present oral airway devices. FIGS. 2A and 2B are front and back views, respectively, thereof. The depicted device has a body 100 that includes a straight section 20, a non-straight (e.g., curved) section 30, a first lumen 40, a second lumen 50, a first connector 60, a second connector 80, a first (ventral) flange portion 110, and a second (dorsal) flange portion 120. In some embodiments, the curved section 30 has a pharyngeal curve. The curved section 30 may be characterized as extending from the straight section 20, and vice-versa. The first lumen 40 and the second lumen 50, which are visible at the distal end 25 of the body 100, are within the straight and curved sections of the body 100. The first connector 60 has a first opening 70 in fluid communication with the first lumen 40. The second connector 80 has a second opening 90 in fluid communication with the second lumen 50. The ventral flange portion 110 extends from a proximal portion of the straight section 20 and the dorsal flange portion 120 extends from the proximal portion of the straight section 20.

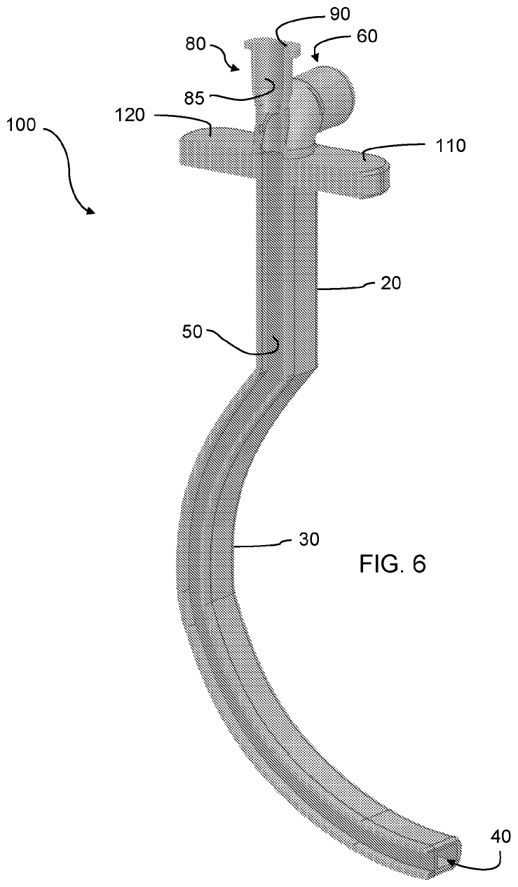

As reflected in these figures and others (such as FIG. 6), the second connector 80 is axially aligned with, and at a minimum is oriented parallel to, the portion of the second lumen 50 within the straight section 20 of body 100. As reflected in these figures and others, the first connector 60 is oriented perpendicular to the straight section 20 of body 100.

FIGS. 3A and 3B are right and left side views, respectively, of the oral airway device depicted in the preceding figures. In FIG. 3A, the first opening 70 of the first connector 60, which can be coupled to an oxygen source, faces the viewer and shows a portion of the first connector 60's lumen, which is in fluid communication with the first lumen 40. FIG. 3A also shows that the length SSL of the straight section 20 of the body 100 is less than the length CSL of the curved section 30 of the body 100. In FIG. 3B, the second connector 80, which can possess any configuration (such as a luer fitting, such as a luer lock fitting or a luer slip (or slip tip) fitting) suitable for coupling the connector to a carbon dioxide sample tube (or line), is visible near the top of the figure.

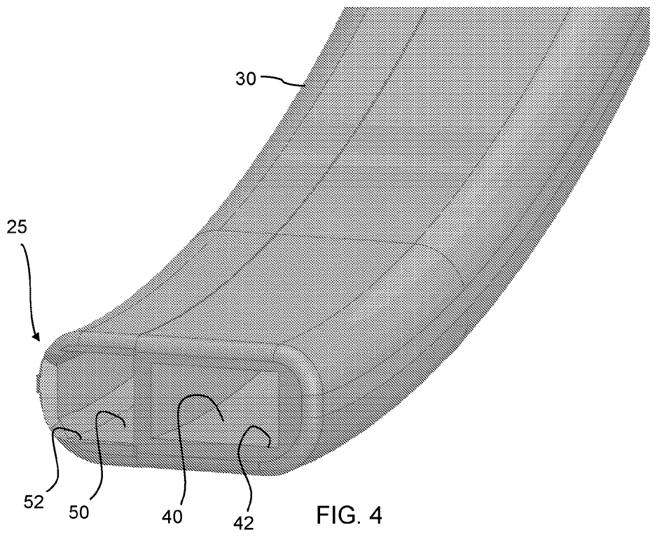

FIG. 4 shows an enlarged detail of a perspective view of the distal end 25 of the body 100 of the depicted oral airway device, showing aspects of the first lumen 40 and the second lumen 50. In at least some embodiments of the present oral airway devices, and as a person of ordinary skill in the art will appreciate from FIG. 4, in a cross-section of the body 100 that intersects and is substantially perpendicular to both the first and second lumens thereof (such as along the straight or curved sections 20 or 30 of the body 100), the first lumen 40 and second lumen 50 are bounded by respective first and second perimeters 42 and 52, with the first perimeter 42 being larger than the second perimeter 52. In at least some embodiments of the present oral airway devices, and as a person of ordinary skill in the art will further appreciate from FIG. 4, the first and second perimeters are non-circular and/or non-oval.

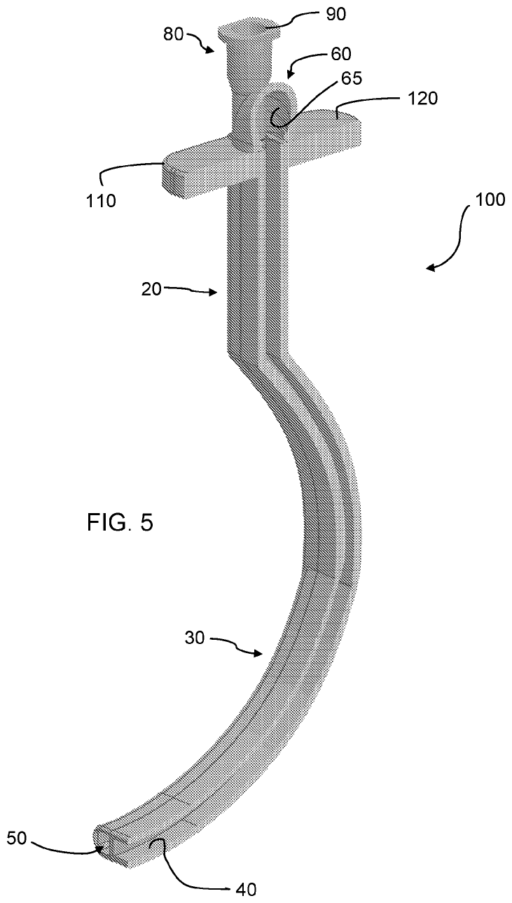

FIG. 5 is a cross-sectional view of the body 100 of the depicted oral airway device, taken along the length of the body 100 and through the first lumen 40, showing the first lumen 40 within the straight section 20 and the curved section 30 of the body. While the size of the first lumen 40 in the ventral-to-dorsal direction varies in the depicted embodiment, it should be understood that, in other embodiments, that size in that direction is substantially unvarying. FIG. 5 also shows a portion of the lumen 65 of the first connector 60 in fluid communication with the first lumen 40.

FIG. 6 is another cross-sectional view of the body 100 of the depicted oral airway device, taken along the length of the body 100 and through the second lumen 50, showing the second lumen 50 within the straight section 20 and the curved section 30 of the body. FIG. 6 also shows that the lumen 85 of the second connector 80 is in fluid communication with the second lumen 50.

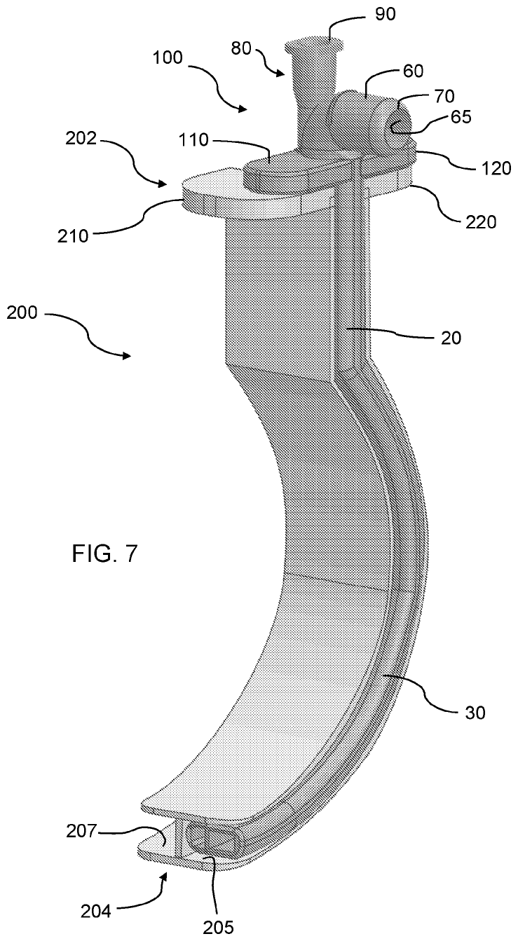

FIG. 7 shows the depicted oral airway device coupled to a host oral airway device 200 having a proximal end 202, a distal end 204, first and second open channels 205 and 207, respectively, extending from the proximal end 202 to the distal end 204, and ventral and dorsal flange portions 210 and 220 extending from a proximal portion of the device 200. In this state, the two devices are ready to be inserted into a patient's mouth; such insertion can be accomplished in any conventional manner that a practitioner would otherwise use to insert into a patient's mouth the host oral airway device by itself. In embodiments of the present kits, the two devices may be in this state within the kit container (e.g., box or pouch). FIG. 7 reflects that the body 100 of the depicted oral airway device is configured so that a portion thereof is insertable into and, while portions of both devices are inserted in a patient's mouth, removable from an open channel (e.g., first open channel 205) of a host oropharyngeal airway device (e.g., host oral airway device 200). In at least some embodiments of the present oral airway devices, and as shown in FIG. 7, the devices may be configured (e.g., sized) such that, when a portion thereof distal to the ventral and dorsal flange portions 110 and 120 is inserted into the first open channel 205 of the host oral airway device 200, some of that portion (e.g., a side) may extend beyond the boundary of the first open channel 205; however, in other embodiments of the present oral airway devices, the devices may be configured so that none of that portion extends beyond the boundary of the open channel in which the portion is inserted. In their coupled state, the ventral and dorsal flange portions 110, 120 of the body 100 are positioned proximal of the ventral and dorsal flange portions 210, 220 of host oral airway device 200; the two airway devices may be configured so that their respective flange portions touch each other in some embodiments when the devices are coupled, while in other embodiments, they may be configured so that their respective flange portions do not touch each other when the devices are coupled. At least a portion of each of the body 100 and the host oral airway device 200 distal to their respective flange portions may be configured to structurally resist damage due to a patient biting down thereon, and thus may each be characterized as having a bite guard.

In at least some embodiments of the present oral airway devices, the body 100 may be unitarily constructed, such as through injection molding, and may be made from any suitable material known to those of ordinary skill in the art, including medical grade plastics and plastic-like materials, such as polypropylene, polyethylene, polycarbonate, polyvinyl chloride, silicone, rubber, urethane, and the like. In at least some embodiments, the material chosen will allow the constructed device to be rigid enough to maintain its shape during use and uncoupling from a host oral airway device (as described below for at least some embodiments) yet flexible enough that the device does not cause undue tissue damage should the device get inadvertently pinched between a laryngoscope (facilitating intubation) and pharyngeal tissue.

Embodiments of the present oral airway devices may be configured with sufficient rigidity and the placement of the flange portions along the length of the device may be such that a practitioner may be able, in some circumstances, to uncouple (or separate) the coupled devices shown in FIG. 7 with one hand. For example, the practitioner may be able to place their index and middle fingers (or their middle and ring fingers) of one hand beneath the ventral and dorsal flange portions 210, 220 of the host oral airway device 200 and, restraining the movement of those flange portions in one lateral direction, push the flange portions 110, 120 and/or some other proximal portion of the body 100 with the thumb of the same hand in an opposing (lateral) direction, thereby uncoupling the devices; as necessary, a practitioner may also use both hands to accomplish this uncoupling.

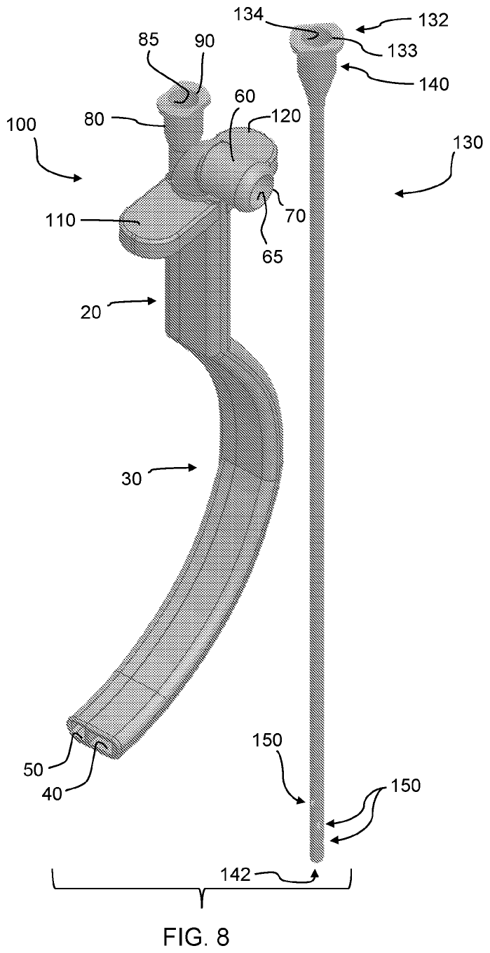

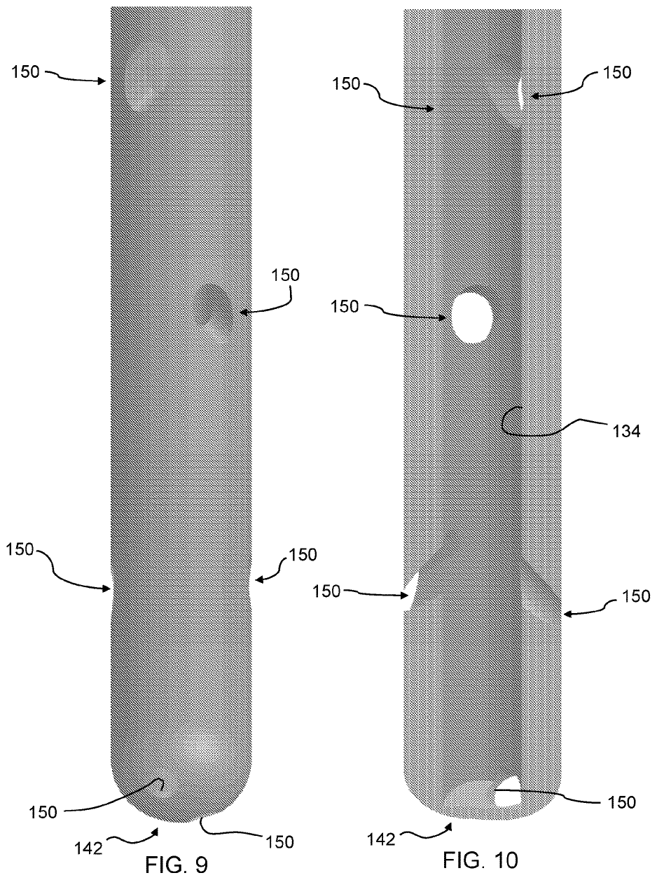

FIG. 8 is a perspective view of the oral airway device depicted in the preceding figures positioned beside spray catheter 130, which is one embodiment of the present spray catheters. The spray catheter 130 has a proximal end 132 with a proximal opening 133, a proximal portion that includes a spray catheter connector 140, a distal portion with multiple openings 150, and a distal end 142. The spray catheter 130 also includes a lumen 134 that extends from proximal opening 133 to the distal end 142, where the lumen 134 terminates due to the distal end 142 of the spray catheter 130 being closed (absent openings 150); the lumen 134 is, however, in fluid communication with the outside of the distal portion of the spray catheter 130 through each opening 150, at least one of which can be positioned at the distal end 142 thereof, as reflected in FIGS. 9-11 and 13.

FIG. 9 is an enlarged detail view of the distal portion of the spray catheter 130 depicted in FIG. 8, showing at least some of the multiple openings 150. In at least some embodiments of the present spray catheters, and as shown in FIG. 9, the multiple openings 150 of the spray catheter 130 can include openings that are spaced axially (or longitudinally) apart from each other along a length (e.g., a one centimeter (cm) length) of the distal portion of the spray catheter 130. Furthermore, in at least some embodiments of the present spray catheters, and as shown in FIG. 9, the multiple openings 150 of the spray catheter 130 can include openings that are also spaced circumferentially apart from each other along a length of the distal portion of the spray catheter 130. The openings 150 may have any suitable size, including 0.40 mm, 0.45 mm, 0.50 mm, 0.55 mm, 0.60 mm, 0.65 mm, 0.70 mm, 0.75 mm, 0.80 mm, and 0.85 mm, which sizes may be characterized as diameters of the openings when the openings are substantially circular in shape, taken across the inner surface defining the lumen 134 of across the outer surface of the spray catheter 130. In addition, any two or more of the different openings among openings 150 may also have different sizes, and the sizes may decrease in the proximal direction of the spray catheter 130 to create a back-pressure gradient that will allow the fluid to fill the spray catheter 130 and spray out of the openings 150 more evenly than if all the openings 150 have the same size.

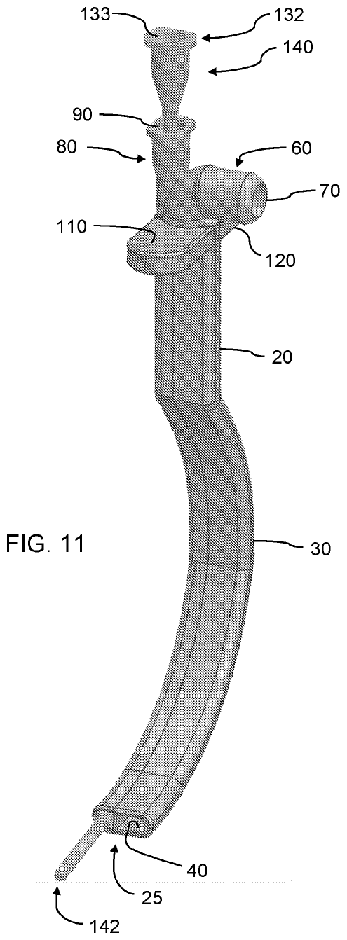

FIG. 10 is a cross-sectional view of a distal portion of the spray catheter 130 depicted in FIG. 8. In this figure, the viewer can see that the lumen 134 is in fluid communication with the exterior of the spray catheter through each of the openings 150. From this figure and others (e.g., FIGS. 8 and 11), it will also be appreciated that when a source (e.g., a syringe, not shown) of local anesthetic (e.g., lidocaine, also not shown) is coupled to connector 140 (which can possess any configuration (such as a luer fitting, such as a luer lock fitting or a luer slip (or slip tip) fitting) suitable for coupling the connector to a syringe or other local anesthetic source), and the local anesthetic is driven from the source and into the spray catheter 130 (while the spray catheter is positioned in the second lumen 50 of the body 100), such anesthetic can be delivered to a patient through the spray catheter 130 by passing through at least a portion of lumen 134 and out of the spray catheter 130 through one or more (e.g., all) of the multiple openings 150, and this can be accomplished blindly, meaning without the use of visualization instrumentation. Furthermore, and as reflected by the positioning of the openings 150, the anesthetic may be sprayed out of the spray catheter 130 in multiple directions at the same time; the openings 150 (which comprise at least two openings) may therefore be referred to as multi-directional openings 150. Furthermore, and as will be appreciated from FIGS. 9 and 10, any one or more of the openings 150 that are proximal of the distal end 142 may be oriented in the wall of the spray catheter 130 at a non-perpendicular angle to an axis centered in the lumen 134 and so that the portion of a given opening bordering the lumen 134 is more proximal than the portion of that opening bordering the outside of the wall of the spray catheter 130; as a result, each such opening passes through the spray catheter wall in a direction that intersects an axis centered in the lumen 134 at a location proximal of the proximal-most portion of that opening bordering the lumen 134.

FIG. 11 is a perspective view showing the body 100 of the depicted oral airway device with the spray catheter 130 inserted into the second lumen 50, which is one way the two devices may be arranged in at least some embodiments of the present kits. This figure reflects that the spray catheter 130 may be made from a suitably flexible material, such as polyurethane, which will allow it to be inserted into and through the second lumen 50 of the body 100 without irreparably damaging itself or the second lumen 50. The spray catheter 130 is configured such that when a portion of the spay catheter 130 is inserted through the second opening 90 and into the second lumen 50, the distal end 142 of the spray catheter 130 is positioned distally of the distal end 25 of the body 100 (which is also the distal end of the second lumen 50) and the spray catheter's proximal portion is positioned proximally of the second connector 80 of the body 100.

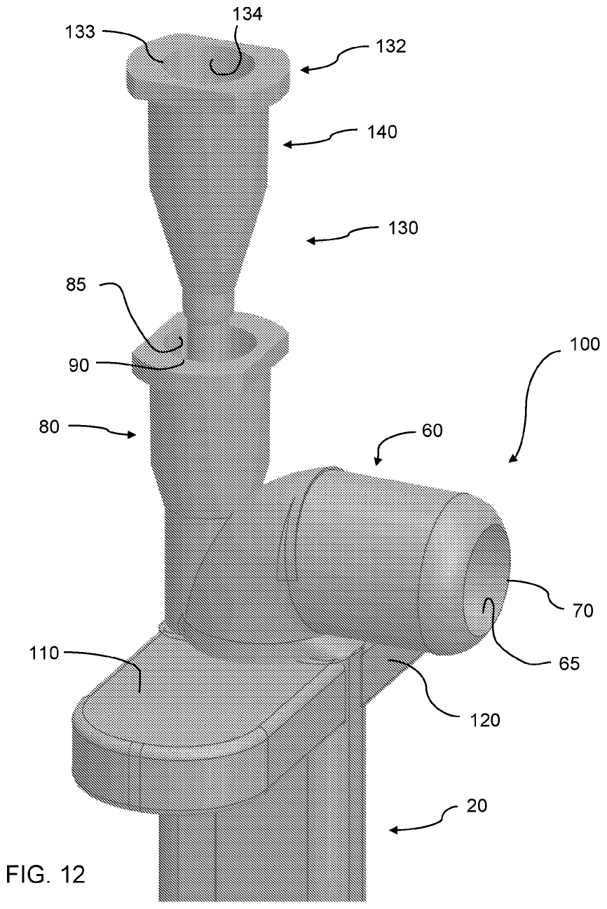

FIG. 12 is an enlarged detail view of the proximal end of the body 100 of the depicted oral airway device, showing also a proximal portion of the spray catheter 130, which has been inserted into the second lumen 50 of the body 100. As FIG. 12 shows, the spray catheter 130 is configured such that when a portion of the spray catheter 130 is inserted through the second opening 90 and into (and through) the second lumen 50, the spray catheter's 130 proximal portion is positioned proximally of the second connector 80 of the body 100. This figure also shows that a proximal portion (and, more specifically, a circumferential aspect) of the spray catheter 130 is larger than the second opening 90, which prevents the spray catheter 130 from falling through the distal end 25 of the body 100.

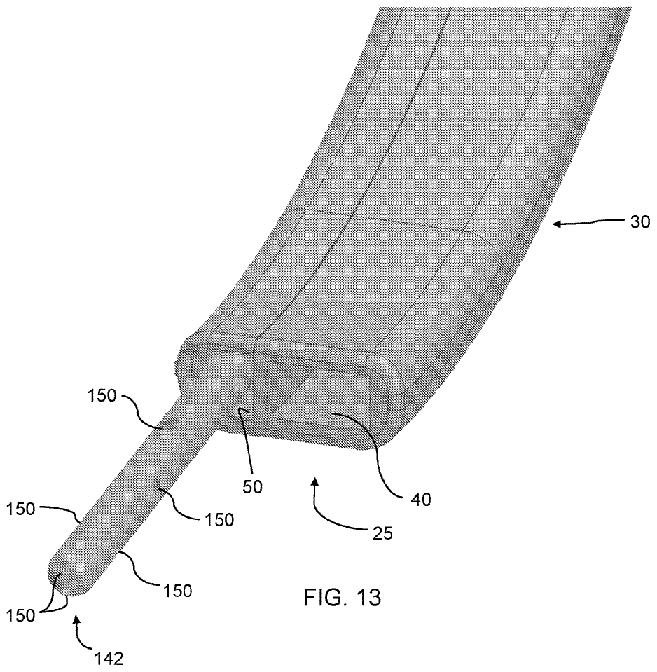

FIG. 13 shows an enlarged detail of a perspective view of the distal end 25 of the body 100 of the depicted oral airway device with the spray catheter 130 inserted through the second lumen 50. As shown, the distal end 142 (and, more specifically, the distal portion with the openings 150) of the spray catheter 130 extends past (and is positioned distally of) the distal end 25 of the body 100. The depicted oral airway device and spray catheter may be sized such that, when the two are arranged as depicted and inserted into a patient's mouth as desired, at least the distal end 142 of the spray catheter 130, and preferably the distal portion that includes the openings 150 of the spray catheter 130, is positioned below the patient's epiglottis.

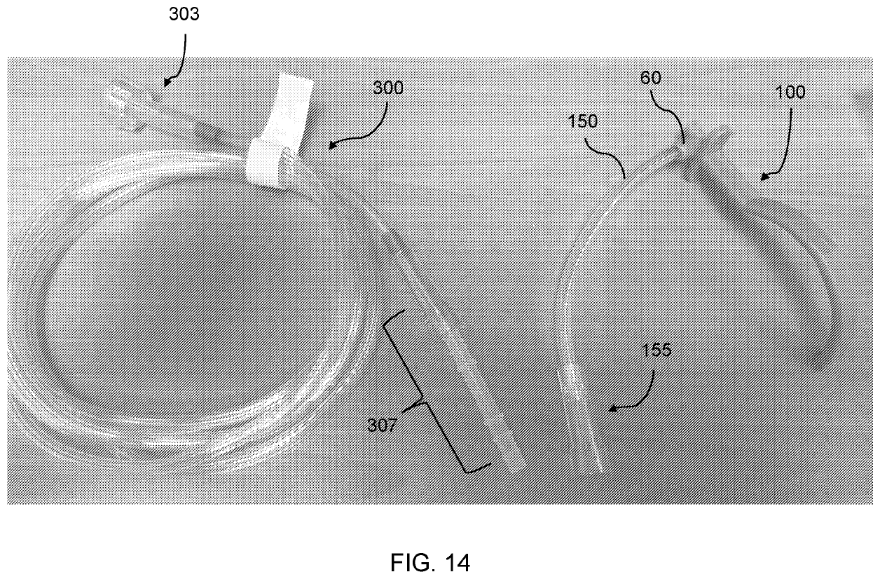

FIG. 14 shows body 100 of the depicted oral airway device with a first length of oxygen tubing 150 connected to the first connector 60, and a second length of oxygen tubing 300 that may be included one of the present kits and used to connect one of the present oral airway devices in such a kit with an oxygen source. For example, second length of oxygen tubing 300 includes a female connector 303 that may be used to connect the tubing 300 to a source of oxygen at a wall or an e-cylinder (not shown), and, at another of its ends, the tubing 300 includes a male connector 307. The first length of oxygen tubing 150 includes a female connector 155 at its free end that may be connected to male connector 307 of tubing 300. The first length of oxygen tubing 150 may have any suitable length, such as 15 to 20 cm in at least some embodiments (including any distance from 15 to 20 cm, including by increments of 1 cm, and shorter or longer distances in other embodiments), and may be connected to the first connector 60 of the body 100 in any suitable way known to a person of ordinary skill in the art, such as through a medical-grade adhesive.

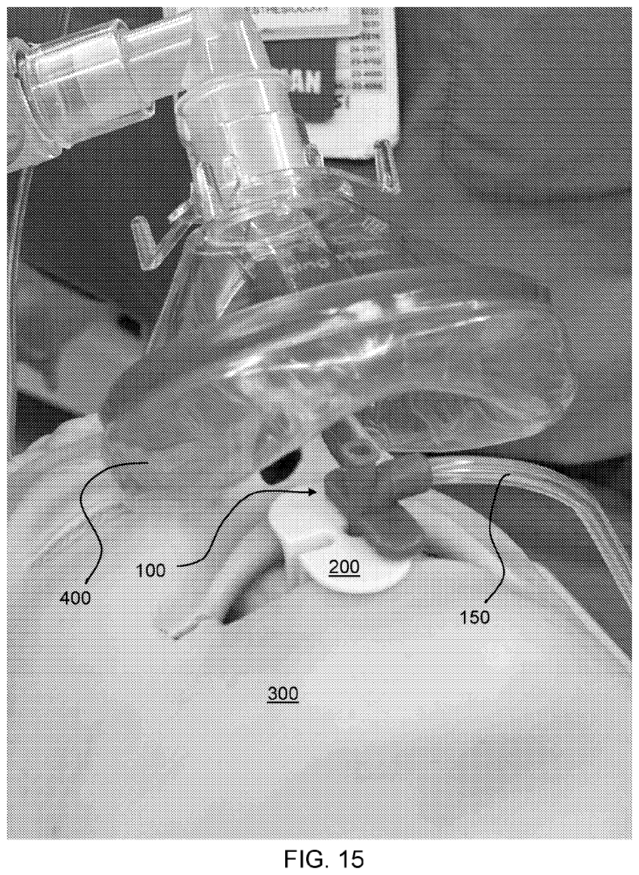

FIGS. 15 and 16 show a mask 400 usable with a bag-valve-mask ventilation system (not shown) being placed into position over the nose and mouth of the head 300 of a mannequin, with body 100 of the depicted oral airway device coupled to the host oral airway device 200 and the length of tubing 150 attached to the first connector (not labeled) of the body 100 prior to sealing the mask over the mannequin's nose and mouth (FIG. 15) and after sealing by a practitioner 500 (FIG. 16). These figures reflect that a mask that is part of a conventional bag-valve-mask system will adequately seal over the nose and mouth of a patient with one of the present oral airway devices inserted in a patient's mouth and connected to a source of oxygen, thus allowing (as in some embodiments of the present methods) a practitioner to ventilate a patient while the patient is receiving oxygen through one of the present oral airway devices.

The above specification and examples provide a complete description of the structure and use of illustrative embodiments of this invention. Although certain embodiments have been described above with a certain degree of particularity, or with reference to one or more individual embodiments, those of ordinary skill in the art could make numerous alterations to the disclosed embodiments without departing from the scope of this invention. As such, the various illustrative embodiments of the present oral airway devices, kits, and methods are not intended to be limited to the particular forms disclosed. Rather, they include all modifications and alternatives falling within the scope of the claims, and embodiments other than those shown may include some or all of the features of the depicted embodiment. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. An oral airway device comprising:
a unitarily-constructed body that includes:
a straight section;
a curved section;
a first lumen within the straight and curved sections of the body;
a second lumen within the straight and curved sections of the body;
a first connector having a first opening in fluid communication with the first lumen; and
a second connector having a second opening in fluid communication with the second lumen.

2. The device of claim 1, wherein the body also includes a ventral flange portion extending from a proximal portion of the straight section, and a dorsal flange portion extending from the proximal portion.

3. The device of claim 2, wherein the first connector is oriented perpendicular to the straight section.

4. The device of claim 3, wherein the second connector is oriented parallel to the straight section.

5. The device of claim 4, wherein in a cross-section of the body taken along the straight section, the first and second lumens are bounded by respective first and second perimeters, and the first perimeter is larger than the second perimeter.

6. The device of claim 4, wherein in a cross-section of the body taken along the curved section in a direction perpendicular thereto, the first and second lumens are bounded by respective first and second perimeters, and the first perimeter is larger than the second perimeter.

7. The device of claim 6, wherein the body is configured so that a portion thereof is insertable into and removable from an open channel of a host oropharyngeal airway device.

8. The device of claim 7, further comprising a length of tubing having a first end that is attached to the first connector and a second end that is attached to a female connector.

9. An oral airway device comprising:
a unitarily-constructed body that includes:
a straight section having a straight section length;
a curved section extending from the straight section, the curved section having a curved section length that is greater than the straight section length;
a first lumen within the straight and curved sections of the body;
a second lumen within the straight and curved sections of the body;
a first connector having a first opening in fluid communication with the first lumen;
a second connector having a second opening in fluid communication with the second lumen;
a ventral flange portion extending from a proximal portion of the straight section; and
a dorsal flange portion extending from the proximal portion.

10. The device of claim 9, wherein the first connector is oriented perpendicular to the straight section.

11. The device of claim 10, wherein the second connector is axially aligned with the portion of the second lumen within the straight section and is also oriented parallel to the straight section.

12. The device of claim 11, wherein in a cross-section of the body taken along the straight section, the first and second lumens are bounded by respective first and second perimeters, and the first perimeter is larger than the second perimeter.

13. The device of claim 11, wherein in a cross-section of the body taken along the curved section in a direction perpendicular thereto, the first and second lumens are bounded by respective first and second perimeters, and the first perimeter is larger than the second perimeter.

14. The device of claim 13, wherein the body is configured so that a portion thereof is insertable into and removable from an open channel of a host oropharyngeal airway device.

15. The device of claim 14, further comprising a length of tubing having a first end that is attached to the first connector and a second end that is attached to a female connector.

16. An oral airway kit comprising:
an oral airway device having a body that includes:
a first lumen;
a second lumen;
a first connector having a first opening in fluid communication with the first lumen; and
a second connector having a second opening in fluid communication with the second lumen; and
a spray catheter having a proximal portion that includes a spray catheter connector and a distal portion with multiple openings, the spray catheter configured such that when a portion of the spray catheter is inserted through the second opening and into the second lumen, a distal end of the spray catheter is positioned distally of a distal end of the second lumen and the spray catheter's proximal portion is positioned proximally of the oral airway device's second connector.

17. (canceled)

18. The kit of claim 16, wherein the oral airway device's body also includes straight and curved sections, the first lumen is positioned within the straight and curved sections, and the second lumen is positioned within the straight and curved sections.

19.-24. (canceled)

25. The kit of claim 18, further comprising:
a host oropharyngeal airway device having a host proximal end, a host distal end, a first open channel extending from the host proximal end to the host distal end, a second open channel extending from the host proximal end to the host distal end, and a rib separating the first and second open channels from each other;
wherein the oral airway device is configured such that a portion thereof is insertable into and, while portions of both the oral airway device and the host oropharyngeal device are inserted in a patient's mouth, removable from the host oropharyngeal device's first open channel.

26. (canceled)

27. An oral airway kit comprising:
an oral airway device having a body that includes:
a first lumen;
a second lumen;
a first connector having a first opening in fluid communication with the first lumen;
a second connector having a second opening in fluid communication with the second lumen;
a ventral flange portion extending from a proximal portion of the body; and
a dorsal flange portion extending from the proximal portion of the body; and
a spray catheter having a proximal portion that includes a spray catheter connector and a distal portion with multiple openings, the spray catheter configured such that when a portion of the spray catheter is inserted through the second opening and into the second lumen, a distal end of the spray catheter is positioned distally of a distal end of the second lumen and the spray catheter's proximal portion is positioned proximally of the oral airway device's second connector.

28.-46. (canceled)
